(19) United States
(12) Patent Application Publication
(10) Pub. No.: US 2013/0083289 A1
(43) Pub. Date: Apr. 4, 2013

(54) Module for Transmitting a Light Beam and Surgical Microscope with Deflecting Element and Fundus Imaging System

(71) Applicant: Carl Zeiss Meditec AG, Jena (DE)

(72) Inventors: Christoph Hauger, Aalen (DE); Artur Hoegele, Oberkochen (DE); Holger Matz, Unterschneidheim (DE); Xing Wei, Dublin, CA (US); Marco Wilzbach, Stuttgart (DE)

(73) Assignee: CARL ZEISS MEDITEC AG, Jena (DE)

(21) Appl. No.: 13/631,411

(22) Filed: Sep. 28, 2012

(30) Foreign Application Priority Data
Sep. 29, 2011 (DE) 10 2011 114 523.4

(51) Int. Cl. A61B 3/13; A61B 3/00

(52) U.S. Cl. CPC A61B 3/13; A61B 3/0008
USPC 351/221

(57) ABSTRACT

The invention is directed to a module for transmitting a light beam (2) from a light source (3) to an object region (4) of a surgical microscope (6). The module (1) has a first interface (5) for attaching the module to the surgical microscope (6) below a main objective (7) of the surgical microscope (6). The module has a second interface (13) for connecting the light source (3) to the module (1) and further includes an imaging optic (18, 19, 20) for imaging the light source (3) in the object region (4) via a scanning beam path (21).

The imaging optic includes a deflecting element (20) in the scanning beam path (21) via which the scanning beam path (21) is deflected in a direction toward the object region (4).

### CROSS REFERENCE TO RELATED APPLICATION

This application claims priority of German patent application no. 10 2011 114 523.4, filed Sep. 29, 2011, the entire content of which is incorporated herein by reference.

### FIELD OF THE INVENTION

The invention relates to a module for transmitting a light beam from a light source to an object region of a surgical microscope. The module comprises a first interface for attaching the module to the surgical microscope below a main objective of the surgical microscope, a second interface for attaching the light source to the module, and an imaging optics for imaging the light source into the object region via a scanning beam path.

The imaging optics includes a deflecting element in the scanning beam path by which the scanning beam path is deflected in the direction of the object region.

The invention further relates to a surgical microscope for ophthalmological applications, having a main objective and having a viewing beam path that passes through the main objective in order to visualize an object region. The surgical microscope further comprises an optical system in which a light beam is guided in a scanning beam path from a light source to the object region, the optical system including a deflecting element in the scanning beam path by which the scanning beam path is deflected in the direction of the object region. Furthermore, the surgical microscope comprises a fundus imaging system for visualizing the fundus of an eye. What is understood in general in this case by a fundus imaging system is an optical system in the viewing beam path of the surgical microscope via which the optical refractive power of the cornea and lens of the eye is compensated, and/or by which an intermediate image of a fundus is formed in cooperation with cornea and lens. Fundus imaging systems can, for example, be designed as a magnifier lens in the viewing beam path (ophthalmoscopic magnifier glass) or as contact lens for placing on the cornea.

### BACKGROUND OF THE INVENTION

The article by Brandenburg, Haller and Hauger entitled: “Real-time in vivo imaging of dental tissue by means of optical coherence tomography (OCT)”, Optics Communications 227 (2003) 203-211 discloses a module via which an OCT system is coupled to a surgical microscope. The module has a first interface via which it is fastened on the surgical microscope below a main objective. A light source in the form of an exit end of a light guide is attached to the module via a second interface. A light beam from the light guide is guided via a collimator lens and a galvoscanner to the object, reflected from there and guided again to the light guide via galvoscanner and collimator lens. A surface of the object to be examined can be scanned in one direction by the light beam with the aid of the galvoscanner. In this case, the galvoscanner is arranged beneath the main objective outside the stereo viewing beam paths of the surgical microscope. It is a disadvantage of this system in that the object to be examined can be scanned by the galvoscanner only in a lateral direction.

U.S. Pat. No. 7,889,423 discloses a surgical microscope into which an illumination module can be inserted. The illumination module comprises an interface for coupling the module to the surgical microscope, and two further interfaces for mounting one light guide each. An illumination beam path can be fed to the illumination module by the first light guide, and an OCT scanning beam path can be fed via the second light guide. Arranged in the module is a beam splitter with the aid of which the illumination beam path and the OCT scanning beam path are superposed when traversing the module. The superposed beam paths are subsequently deflected by deflecting element in the direction of the object region, and guided through a main objective to the object outside the viewing beam paths of the surgical microscope. The illumination module is designed so that it can be introduced above the objective into a basic body of the microscope. A complicated receiving device for the illumination module needs to be provided for this purpose on the basic body of the microscope. A standard surgical microscope cannot be retrofitted with the illumination module of United States Patent 7,889,423.

U.S. Pat. No. 7,978,404 discloses superposing an OCT scanning beam path on an viewing beam path for an assistant. For this purpose, U.S. Pat. No. 7,978,404 makes recourse to an integral arrangement of the OCT system in the surgical microscope.

U.S. Pat. No. 7,901,080 discloses a surgical microscope with an OCT system and a fundus observation system. The OCT scanning beam path is guided to an object in this case through a main objective of the surgical microscope, a reducing lens and an ophthalmoscopic magnifier lens. Here, as well, the OCT system is an integral component of the surgical microscope, and so no retrofitting is possible.

United States patent application publication 2003/0218755 A1 discloses a surgical microscope for ophthalmological applications that has a main objective, an viewing beam path for visualizing an object region penetrating the main objective. The surgical microscope has an optical system in which a beam is guided from an OCT unit into a scanning beam path. The optical system comprises a deflecting element by which the scanning beam path is deflected in the direction of the eye to be examined.

### SUMMARY OF THE INVENTION

An object of the invention consists in providing a module by which a surgical microscope can be retrofitted flexibly with one or more additional optical examination systems.

The module of the invention is for transmitting a light beam from a light source to an object region of a surgical microscope having a main objective. The module is adapted to coact with a fundus imaging system and includes: a first interface for attaching the module to the surgical microscope below the main objective thereof; a second interface for attaching the light source to the module; an imaging optic for imaging the light source in the object region via a scanning beam path; the imaging optic including a stationary deflecting element arranged in the scanning beam path for deflecting the scanning beam path in a direction toward the object region; the imaging optic including a scanning unit disposed in the scanning beam path between the second interface and the deflecting element; and, a third interface arranged in the scanning beam path downstream of the deflecting element for attachably connecting the fundus imaging system to the module.

According to the invention, the deflecting element is of stationary design, and the module comprises a scanning device in the scanning beam path of the module between the second interface and the deflecting element, and the module has a third interface that is arranged in the scanning beam path downstream of the deflecting element and by which a fundus imaging system can be linked to the module. The deflecting element is therefore arranged immovably relative to the module and—if the module is fastened on the surgical microscope—relative to the surgical microscope. Consequently, the position of the deflecting element relative to the viewing beam paths of the surgical microscope also remains unchanged irrespective of the current position of the scanning device and irrespective of the type of an optical examination system connected to the light guide. The scanning device in the scanning beam path between the second interface and the deflecting element enables the light beam to be moved over a surface of the object to be examined, and thus enables the object to be scanned. In combination, the invention permits an object scan to be carried out without influencing the viewing beam paths by movements of the scanning device. Here, a fundus imaging system is understood to be an optical system for ophthalmological applications that comprises at least one refractive optical element which is introduced into the viewing beam path of a surgical microscope upstream of the eye to be examined. The refractive optical element produces an intermediate image of the fundus of the eye onto which the main objective of the surgical microscope is focused. The fundus of the eye can be viewed in this way with the surgical microscope. The additional linking of the fundus imaging system to the module permits an application of the additional optical examination system to a fundus of the eye.

In one embodiment of the invention, the scanning device comprises a first scanning mirror, which is supported such that it can pivot about a first axis. This provides a particularly simple embodiment of the scanning device.

In a further embodiment of the invention, the first scanning mirror is supported such that it can pivot about a second axis. The second axis is in this case arranged at an angle to the first axis, preferably orthogonally to the first axis. Scans can therefore be carried out in two mutually independent directions.

In a further embodiment of the invention, the scanning device comprises a second scanning mirror, which is supported such that it can pivot about a third axis. It is preferred in this case to arrange the third axis to differ from the first axis of the first scanning mirror. The third axis is preferably designed to be skewed or orthogonal to the first axis. The second scanning mirror can likewise be used to carry out scans in two mutually independent directions. At the same time, the two scanning mirrors enable a greater flexibility with regard to the beam guidance of the scanning beam in the module. For example, the two scanning mirrors can be arranged in the module so that the scanning beam leaves the scanning device approximately in the same direction in which it was introduced into the scanning device.

In a further embodiment of the invention, the deflecting element is designed as a dichroic beam splitter plate or as a glass block with integrated dichroic beam splitter, or as a mirror. By means, in particular, of the two first named embodiments, it is possible to place the deflecting element in an viewing beam path, or so as to be partially covered by an viewing beam path of the surgical microscope so that the scanning beam path can be at least partially superposed on the viewing beam path.

A further object of the invention consists in providing a surgical microscope for ophthalmological applications that comprises an optical system for providing a light beam, and that it is distinguished by a particularly simple assembly.

The surgical microscope of the invention is for ophthalmological applications. The surgical microscope includes: a main objective; an optical viewing system defining a viewing beam path passing through the main objective for visualizing an object region; a light source; an optical system defining a scanning beam path for transmitting a light beam from the light source along the scanning beam path; the optical system including a deflecting element disposed in the scanning beam path for deflecting the scanning beam path in a direction toward the object region; a fundus imaging system for visualizing a fundus of an eye; the deflecting element and the fundus imaging system being arranged between the main objective and the object region; and, the deflecting element being disposed in the viewing beam path between the main objective and the fundus imaging system.

According to the invention, the deflecting element and the fundus imaging system are arranged between the main objective and the object region, and the deflecting element is arranged in the viewing beam path between the main objective and the fundus imaging system. At this location, the deflecting element or the optical system connected thereto and the fundus imaging system can be fastened in a simple way on the surgical microscope, or be integrated in the surgical microscope, without the need for major structural alterations or adaptations to be applied to the surgical microscope. Owing to the arrangement of the deflecting element in the viewing beam path between the main objective and the fundus imaging system, the surgical microscope can be configured so that the scanning beam path traverses the fundus imaging system and is directed or focused onto the fundus of the eye.

In one embodiment of the invention, the optical system is designed as an OCT system or as a laser system. In this case, an OCT system is understood as a system for carrying out optical coherence tomography (OCT). It is an essential feature of the optical system that a light beam in the form of a laser beam is guided to the object region by the deflecting element arranged below the main objective.

In a further embodiment of the invention, the surgical microscope comprises a reducing optics which is arranged in the viewing beam path of the surgical microscope, and the deflecting element of the optical system is arranged between the reducing optics and the fundus imaging system. In this case, a reducing optics is understood as an optical element that is arranged in the viewing beam path, the combination of reducing optics and main objective having, in contrast to the main objective alone, a changed, in particular shortened focal length. Having the arrangement of a reducing optics in the viewing beam path, it is possible to focus the surgical microscope onto another plane, for example an intermediate image plane, without having in this case to substantially alter a working distance between the main objective and the object. Owing to the arrangement of the deflecting element between the reducing optics and the fundus imaging system, the imaging beam path through the reducing optics is not influenced.

In a further embodiment of the invention, the fundus imaging system and the optical system with the deflecting element are arranged in a common module. With particular preference, the common module can optionally be introduced into the viewing beam path or be removed from the viewing beam path. This provides a surgical microscope with compact dimensions that can be used with particular versatility.

### BRIEF DESCRIPTION OF THE DRAWINGS

The invention will now be described with reference to the drawings wherein:

FIG. 1 shows a schematic of an inventive module on a surgical microscope;

FIG. 2 shows a schematic of a linkage of various optical systems to the module;

FIG. 3 shows a schematic of a combination of a surgical microscope, module and fundus observation system;

FIG. 4 shows the combination from FIG. 3, supplemented with reducing optics;

FIG. 5 shows the combination from FIG. 4 with an alternative arrangement of the components;

FIG. 6 shows the combination from FIG. 4 with a detailed illustration of a fundus observation system; and,

FIG. 7 shows the combination of a surgical microscope, module and an alternative fundus observation system.

### DESCRIPTION OF THE PREFERRED EMBODIMENTS OF THE INVENTION

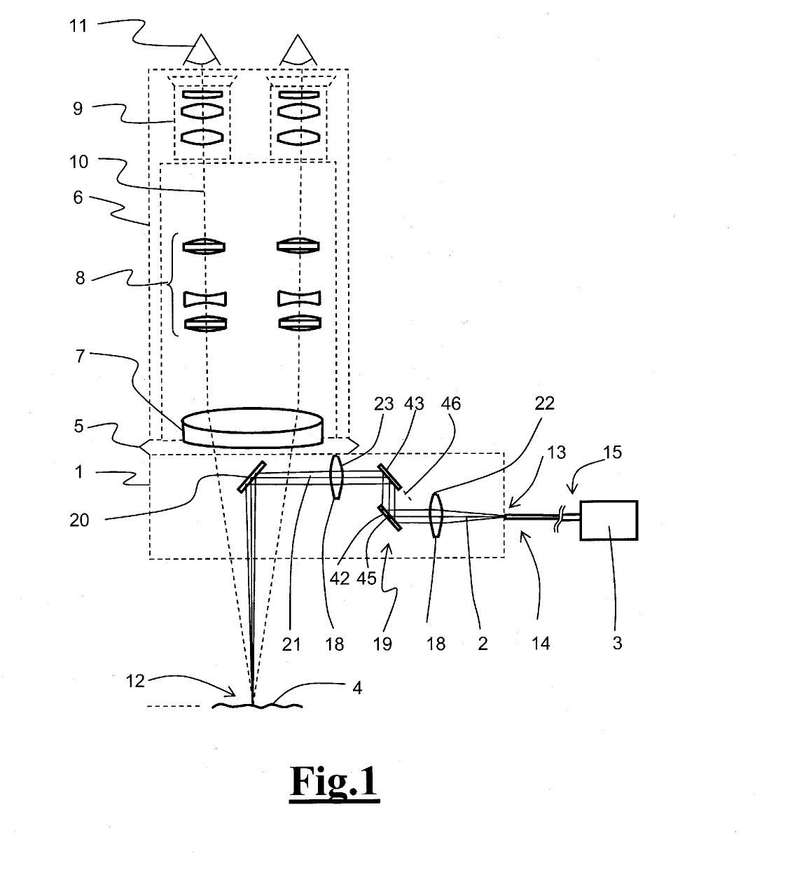

FIG. 1 illustrates an inventive module 1 for transmitting a light beam 2 from a light source 3 to an object region 4. The module 1 is coupled to a surgical microscope 6.

In this embodiment, the surgical microscope has a zoom system 8 and an eyepiece 9 which are respectively constructed from a plurality of individual lenses. The main objective 7, the zoom system 8 and the eyepiece 9 define an viewing beam path 10 such that an observer 11 can view an image of an object 12 through the surgical microscope 6. In FIG. 1, the surgical microscope is designed as a stereomicroscope having an viewing beam path for the left eye and an viewing beam path for the right eye of the observer. However, the invention is not limited to stereomicroscopes, but can also be applied easily to monoscopic microscopes.

The module 1 is connected to the surgical microscope 6 via a first interface 5. The first interface 5 is preferably designed as a standard interface, for example as a dovetail connection, so that instead of the module 1, it is also possible to fasten any other desired accessory parts or examination devices to the first interface 5 of the surgical microscope 6.

The module 1 further comprises a second interface 13 to which it is possible to connect a fiber end, designed as exit end 14, of an optical fiber 15. A light source 3 is arranged at an entry end of the optical fiber 15. Light from the light source 3 is transmitted to the exit end 14 through the optical fiber 15. The light source 3 can, for example, be designed as a laser or as a light emitting diode. However, other light sources, such as xenon lamps or halogen lamps, are also conceivable without limitation of the generality.

In an alternative exemplary embodiment (not shown), the light source is arranged directly on the second interface without interposition of an optical fiber, and so the light from the light source can be coupled into the module without interposition of an optical fiber.

The module 1 comprises a lens system 18, a scanning device 19 and a deflecting element 20, which together form an imaging optic. When the module is coupled to the surgical microscope, the exit end 14 of the optical fiber 15 is imaged into the object region 4 through the imaging optics. The beam path through the module 1 to the object region 4 is designated as scanning beam path 21.

A first lens 22 of the lens system 18 serves to collimate the portion of the scanning beam path 21 exiting on the exit end 14 of the optical fiber 15. The scanning beam path 21 can be focused onto the object region 4 with the aid of a second lens 23 of the lens system 18.

Arranged downstream of the first lens 22 is a scanning device 19 which has a first scanning mirror 42 and a second scanning mirror 43 in the scanning beam path. The first scanning mirror 42 comprises a first pivot axis 45, which is aligned perpendicular to the plane of the drawing in FIG. 1. The first scanning mirror 42 is mounted such that it can pivot relative to a basic body of the module 1 via the pivot axis 45. The second scanning mirror 43 has a second pivot axis 46 via which the second scanning mirror is likewise supported such that it can pivot on the basic body of the module 1, and which is arranged in the plane of the drawing in FIG. 1. The first pivot axis 45 and the second pivot axis 4b are thus aligned orthogonal to one another. The scanning beam path 21 can be guided for scanning over a surface of the object 12 by pivoting the two pivot axes.

Arranged in the further course of the scanning beam path 21 is a deflecting element 20 that is designed in this embodiment as a mirror, and is arranged in the module 1 outside the viewing beam path 10. In this case, the deflecting element 20 is connected to a basic body of the module 1, and thus is designed to be immovable or stationary relative to the module 1 and to the surgical microscope 6 when the module is coupled on.

In a further embodiment (not shown), the deflecting element is designed as a dichroic beam splitter plate or as a glass block, and is arranged in the module in such a way that the deflected scanning beam path is fully or partially superposed on the viewing beam path of the surgical microscope when the module is coupled on. Consequently, the scanning beam path is guided virtually coaxially with the viewing beam path. When the module is applied in ophthalmology, shading effects in the region of an iris of the eye are minimized in this way.

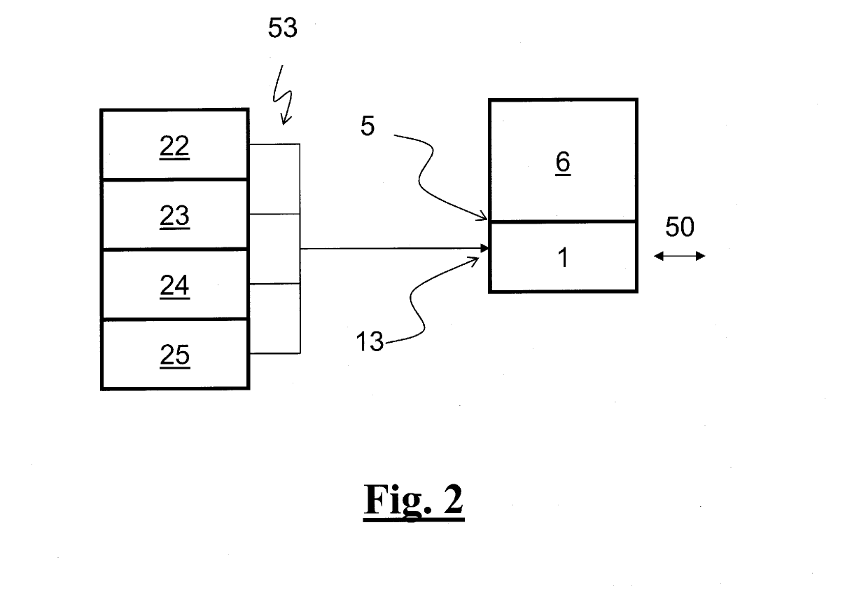

FIG. 2 shows a schematic of combination options of the inventive module 1 for transmitting a light beam with the aid of varied medical devices. The inventive module 1 for transmitting a light beam may be coupled via the second interface 13 to, for example, light outputs 53 of a module for optical coherence tomography (designated below as OCT module 22), of a module for wavefront analyses (designated below as wavefront module 23), of a laser therapy module 24 and/or of a laser vibrometer 25. All of the above-mentioned medical devices have in common that their function is based on a light beam (as a rule, a laser beam) that is guided over a surface of an object that is to be examined or treated. At least in the cases of the OCT module 22 and the wavefront module 23, a reflected light beam is also guided back from the object through the module 1 to the OCT module 22 and/or the wavefront module 23, and analyzed there.

The module 1 is preferably designed such that it can be swung or pushed into the viewing beam path of the surgical microscope 6 so that it can be introduced into the viewing beam path 10 if required. If the medical device (22, 23, 24, 25) connected to the module 1 via the second interface 13 is no longer required, the module 1 can easily be removed out from the viewing beam path 10 of the surgical microscope 6.

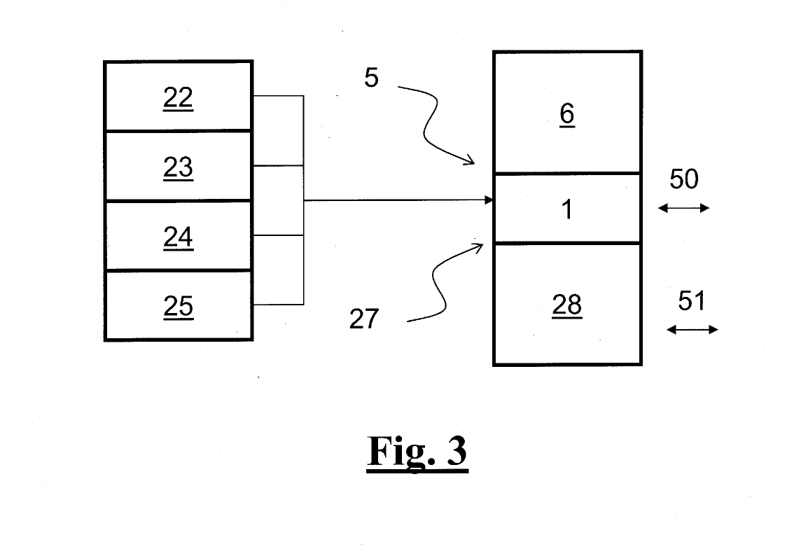

FIG. 3 is a schematic of a module 1 that is coupled to the surgical microscope 6. A fundus observation system 28 for ophthalmological applications is arranged on the module 1 via a third interface 27. The third interface 27 is in this case preferably designed as a standard interface, for example as a dovetail connection, and so it is also optionally possible to fasten other medical devices or accessory parts on the module 1. The fundus observation system 28 enables an observation of the fundus of the eye through the surgical microscope and, for example, comprises an ophthalmoscopic magnifier lens 47 or a contact lens 54.

As shown in FIG. 3 with double arrows (50, 51), the module 1 and the fundus observation system 28 are preferably designed such that they can be swung or pushed or otherwise introduced into the viewing beam path jointly or alternatively.

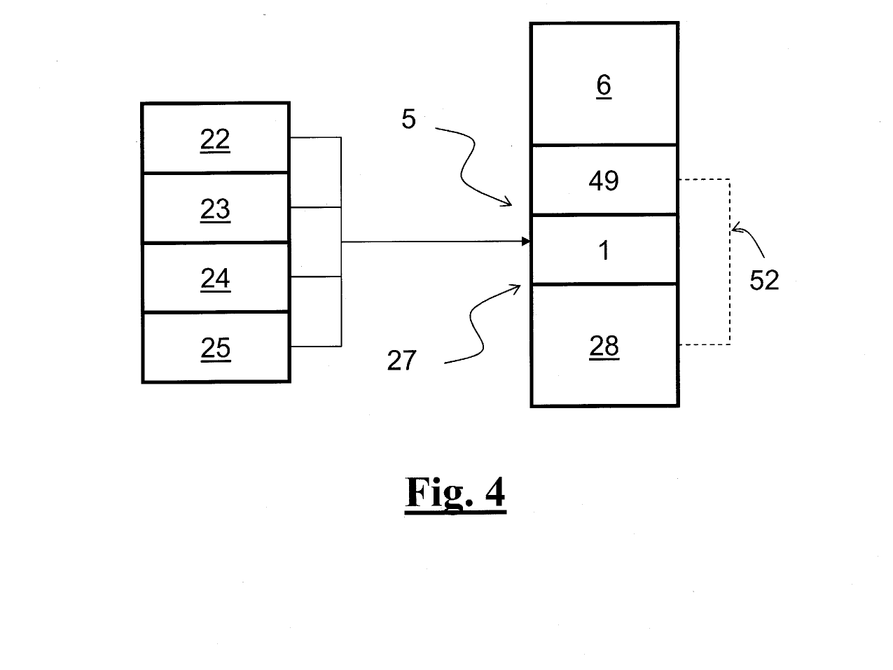

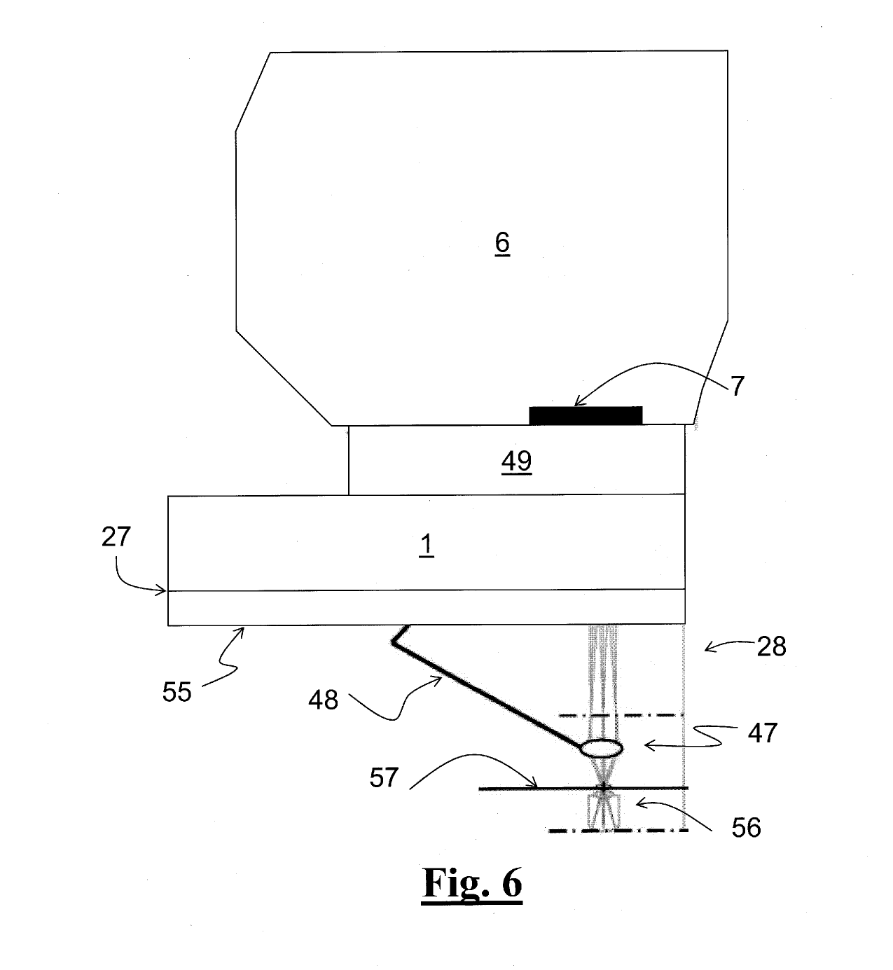

The design in the schematic of FIG. 4 differs from the embodiment of FIG. 3 in that additionally present is a reducing optics 49 that can optionally be introduced into the viewing beam path of the surgical microscope between the main objective 7 and the module 1. The reducing optics 49 comprises at least one optical element, for example a lens, that forms together with the main objective 7 an optical systems whose focal length is less than the focal length of the main objective alone. The use of a reducing optics 49 is particularly to be advised when, as fundus observation system, an ophthalmoscopic magnifier lens 47 (see FIG. 6) is provided, that can be used to produce an intermediate image of the fundus of the eye. By jointly swinging the reducing optics 49 and the ophthalmoscopic magnifier lens 47 into and out of the viewing beam path 10 of the surgical microscope 6, it is easily possible to switch to and fro between viewing the fundus of the eye and viewing another section of the eye (for example the cornea), without the need to refocus the surgical microscope 6 to a significant extent. It is preferred to this end to provide a mechanical, electrical or some other design of coupling 52 between the reducing optics 49 and fundus observation system 28 which ensures that the reducing optics 49 and fundus observation system 28 can be jointly introduced into the viewing beam path of the surgical microscope.

In an alternative embodiment (not shown), the inventive module is integrated in a fundus observation system of the type mentioned above and designed as one component.

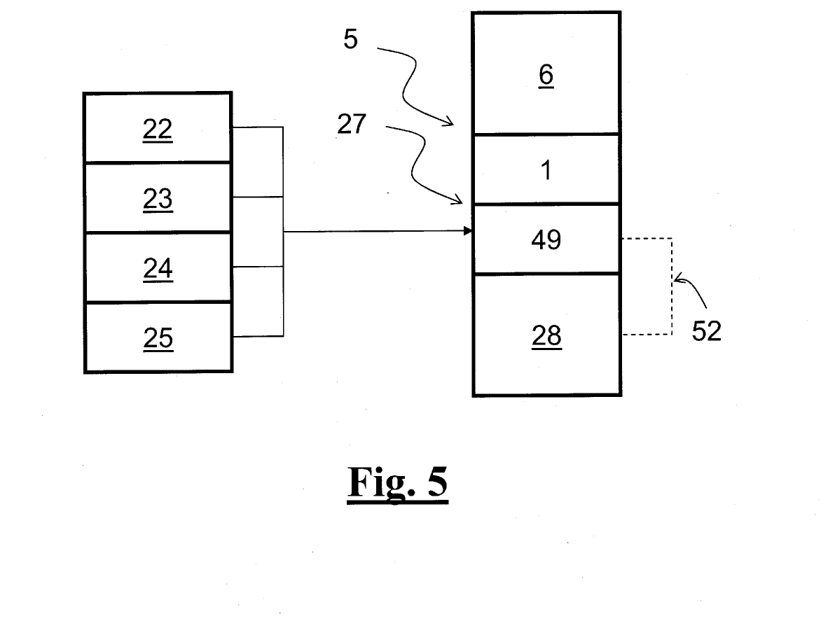

The embodiment shown in FIG. 5 differs from the embodiment in accordance with FIG. 4 in an alternative arrangement of the components. In FIG. 5, the reducing optics 49 is arranged in the beam path between the module 1 and the fundus observation system 28.

In an embodiment illustrated in FIG. 6, the fundus observation system comprises an ophthalmoscopic magnifier lens 47 which is connected to a basic body 55 of the fundus observation system 28 via a bipartite support arm 48 which basic body is, in turn, coupled to the module 1 via the third interface 27. The ophthalmoscopic magnifier lens 47 can be introduced into the viewing beam path 10 of the microscope near the eye to be examined. The lens of the eye and the cornea of the eye 56 to be examined, and the ophthalmoscopic magnifier lens 47 together form an optical system by which an intermediate image of the fundus of the eye is produced in an intermediate plane 57. By introducing the reducing optics 49 into the viewing beam path, the focal length of the main objective 7 is reduced so that the surgical microscope 6 is focused onto the intermediate image plane 57.

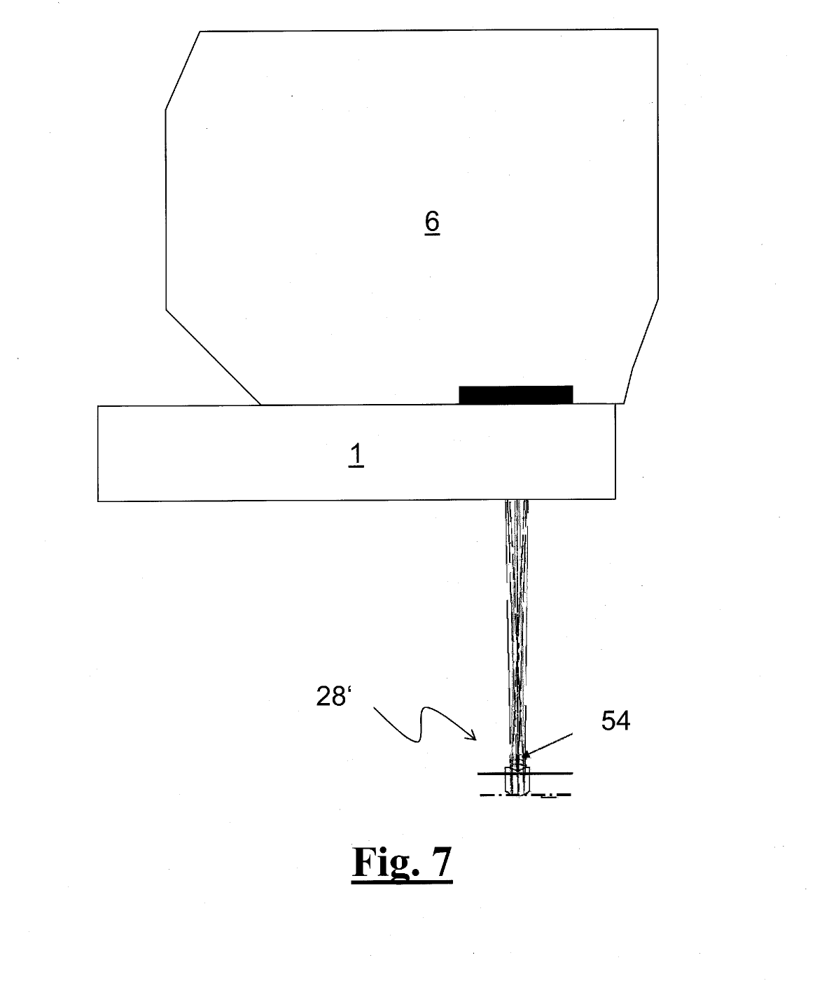

In an alternative exemplary embodiment, illustrated in FIG. 7, the fundus observation system 28′ comprises a contact lens 54 which can be mounted on the cornea of the eye. The optical refractive power of the lens of the eye and cornea is thereby compensated, so that the observer can recognize the fundus of the eye through the surgical microscope.

In summary, it can be seen that a first embodiment of the invention provides that the deflection element 20 is configured to be stationary and the module 1 includes a scanning device 19 in the scanning beam path 21 between the second interface 13 and the deflecting element 20. The module 1 further includes a third interface 27 which is disposed in the scanning beam path 21 after the deflecting element 20. A fundus imaging system 28 can be attached to the module 1 via the third interface 27.

Another embodiment of the invention is also directed to a surgical microscope for ophthalmological applications. The surgical microscope includes a main objective 7 and a viewing beam path 10 which passes through the main objective 7. An optical system (22, 23, 24, 25) is provided wherein a light beam 2 is guided in a scanning beam path 21 from a light source 3. The optical system includes a deflecting element 20 in the scanning beam path 21 via which the scanning beam path 21 is deflected in a direction toward an object region 4 of the surgical microscope. The surgical microscope further includes a fundus imaging system 28.

According to a feature of the invention, the deflecting element 20 and the fundus imaging system 28 are arranged between the main objective 7 and the object region 4 and the deflecting element 20 is arranged in the viewing beam path 10 between the main objective 7 and the fundus imaging system 28.

It is understood that the foregoing description is that of the preferred embodiments of the invention and that various changes and modifications may be made thereto without departing from the spirit and scope of the invention as defined in the appended claims.

What is claimed is:

1. A module for transmitting a light beam from a light source to an object region of a surgical microscope having a main objective, the module being adapted to coact with a fundus imaging system and comprising:
a first interface for attaching said module to said surgical microscope below said main objective thereof;
a second interface for attaching said light source to said module;
an imaging optic for imaging said light source in the object region via a scanning beam path;
said imaging optic including a stationary deflecting element arranged in said scanning beam path for deflecting said scanning beam path in a direction toward said object region;
said imaging optic including a scanning unit disposed in said scanning beam path between said second interface and said deflecting element; and,
a third interface arranged in said scanning beam path downstream of said deflecting element for attachably connecting said fundus imaging system to said module.

2. The module of claim 1, wherein said scanning unit includes a first scanning mirror pivotally journalled about a first axis.

3. The module of claim 2, wherein said first scanning mirror is pivotally journalled about a second axis.

4. The module of claim 2, wherein said scanning unit includes a second scanning mirror pivotally journalled about a third axis.

5. The module of claim 1, wherein said deflecting element is configured as a dichroic beam splitter plate.

6. The module of claim 1, wherein said deflecting element is configured as a glass block having an integrated dichroic beam splitter.

7. The module of claim 1, wherein said deflecting element is configured as a mirror.

8. A surgical microscope for ophthalmological applications, the surgical microscope comprising:
a main objective;
an optical viewing system defining a viewing beam path passing through said main objective for visualizing an object region;
a light source;
an optical system defining a scanning beam path for transmitting a light beam from said light source along said scanning beam path;
said optical system including a deflecting element disposed in said scanning beam path for deflecting said scanning beam path in a direction toward said object region;
a fundus imaging system for visualizing a fundus of an eye;
said deflecting element and said fundus imaging system being arranged between said main objective and said object region; and,
said deflecting element being disposed in said viewing beam path between said main objective and said fundus imaging system.

9. The surgical microscope of claim 8, wherein said optical system is configured as an OCT-system.

10. The surgical microscope of claim 8, wherein said optical system is configured as a laser system.

11. The surgical microscope of claim 8, wherein said deflecting element is disposed in said viewing beam path between said main objective and said fundus imaging system.

12. The surgical microscope of claim 11, further comprising a reducing optic arranged in said viewing beam path; and, said deflecting element of said optical system being disposed between said reducing optic and said fundus imaging system.

13. The surgical microscope of claim 8, wherein said fundus imaging system and said optical system are arranged in a common module.
